(19) United States
(12) Patent Application Publication
(10) Pub. No.: US 2005/0090480 A1
(43) Pub. Date: Apr. 28, 2005

(54) Use of selected amino acid-zinc complexes as anti-malarials

(75) Inventors: Pawan Malhotra, New Delhi (IN); Palakodeti Venkata Naga Dasaradhi, New Delhi (IN); Asif Mohammed, New Delhi (IN); Manzar Jamal Hossain, New Delhi (IN); Sunil Mukherji, New Delhi (IN); Manivel Venkatasamy, New Delhi (IN); Kanury Venkata Subba Rao, New Delhi (IN); Gyan Chandra Mishra, Pune (IN); Parameswaran Perunninakulath Subrayan, Dona Paula (IN); Anil Chatterji, Dona Paula (IN)

Correspondence Address: WOMBLE CARLYLE SANDRIDGE & RICE, PLLC, P.O. BOX 7037, ATLANTA, GA, 30357-0037, US

(73) Assignee: Council of Scientific & Industrial Research, Rafi Marg (IN)

(21) Appl. No.: 10/893,531

(22) Filed: Jul. 19, 2004

Related U.S. Application Data
(60) Provisional application No. 60/512,778, filed on Oct. 21, 2003.

(51) Int. Cl. A61K 31/555; A61K 31/315; A61K 31/47

(52) U.S. Cl. 514/184; 514/494; 514/313; 514/305

(57) ABSTRACT

The invention provides the use of zinc complexes of selected amino acids from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc. The use of the compound comprises administering an effective amount of said compounds for inhibition of growth: of the malarial parasite, Plasmodium falciparum. The compound is lethal to the parasite in RBC cultures but have no effect on the RBCs. The compound has also displayed activity against, the chloroquine-resistant strain-W2Mef. The dose response curves for both 3D7 and W2Mef strains are identical which strongly suggested that the compound is equally effective against field isolates of chloroquine-resistant, P. falciparum. The compound acts on W2Mef strain through killing the target.

### FIELD OF THE INVENTION

The invention provides the use of zinc complexes of selected amino acids wherein the amino acids are selected from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc. The use of the compound comprises administering, an effective amount of said compounds for inhibition of growth of the malarial parasite, Plasmodium falciparum. These compounds are lethal to the parasite in RBC cultures but have no effect on the RBCs. The compound has also displayed activity against the chloroquine-resistant strain-W2Mef. The dose response curves for both 3D7 and W2Mef strains are identical which strongly suggested that the compound is equally effective against field isolates of chloroquine-resistant, P. falciparum. The compound acts on W2Mef strain through killing the target.

### BACKGROUND OF THE INVENTION

Malaria is an important tropical parasitic disease. Relatively it kills more people than any other communicable diseases except tuberculosis. About 300-400 million people come in contact with the malarial parasite every year and about 1.7 million death have been reported every year due to malaria. Malaria represents the toughest challenge facing modern medicine as parasite has a complex life cycle involving two hosts, human and mosquito and there is no malaria vaccine yet in sight. In developing countries, specially, in Africa, malaria leads to enormous loss of human lives and serious economic and medical costs. The causative agents in humans are four species of single-celled parasites, borne by mosquitoes. Among these, the parasite, P. falciparum accounts for majority of the lethal infections.

The Current Global Picture

Today, malaria has become a serious public health problem in more than 90 countries inhabited by a total of some 2,400 million people (about 40% of the worlds population). Worldwide prevalence of the disease is estimated to be of the order of 300-500 million clinical cases every year. More than 90% of all malaria cases are in sub-Saharan Africa. Mortality due to this disease is estimated to be over 1 million each year. The vast majority of these deaths occur among young children in Africa, especially in remote rural areas with poor access to health services. With the development and spread of resistance to most of current anti-malarials, there is a need to develop new anti malarials.

Current Status of Anti-malarial Drugs

Chloroquine (a rapid schizonticide against all infections of Plasmodium malariae and P. ovale as well as immature gametocytes of P. falciparum and not active against intrahepatic forms), Amodiaquine (an antipyretic and anti-inflammatory drug and effective than chloroquine in cleaning the parasites from the blood to enhance clinical recovery faster), Sulfadoxine—Pyrimethamine (highly active blood schizonticides against P. falciparum and less effective against other Plasmodium sp) Proguanil (a synthetic biguanide derivative of pyrimidine with marked effect on the primary tissue stages of P. falciparum, P. vivax and P. ovale), Mefloquine (a potent long acting blood schizonticide against. P. falciparum and highly active against P. vivax and P. malariae. It is not gamatocyiocidal and not active against the hepatic stages of malarial parasites), Quinine (a reasonable option against P. falciparum resistant to chloroquine and Sulfadrug—pyrimethamine combinations. It is extensively used in South East Asia), Halofantrine (a schizonticide that is active against all malarial parasites especially to chloroquine and sulphadrug resistant P. falciparum. It has no place in malarial control because of its high cost, variable bioavailability and cardio toxicity), Artemisinin, (isolated from Artemisia annua in China and effective against P. vivax and chloroquine and Sulphadrug—pyrimethamine resistant P. falciparum. Artemisinin and its derivatives Artemether and Artesunata are the most rapidly effective antimalarial drugs).

Indian green mussels (Perna viridis) are a cheap source of proteins and considered as a delicacy. Extracts prepared from green mussels by enzyme-acid hydrolysis process have shown various biological activities including antimalarial activity. Attempts have been made to purify the active antimalarial compound that showed inhibition of growth of malarial parasite (Plasmodium falciparum and P. berghei). Purification from the crude extract was achieved using a combination of chromatographic methods such as HPLC, gel filtration and TLC. An activity-guided fractionation strategy was pursued until the active principle was purified to homogeneity, and its structure determined. The purified compound was confirmed to retain the above activity. The compound was characterized using NMR and LC-MS/MS techniques. The active compounds were synthesized using known methods and validated for the biological activity. This patent in particular describes the compound and their anti-malarial activity.

Zinc ions are an essential element, found in every cell in the body. They play a vital role in the regulation and catalytic activity of biological systems. It is an integral component of over 200 enzymes and is highly concentrated in red and white blood cells. It regulates various hormonal activities such as growth and sex hormones.

Chloroquine Resistance

Resistance of P. vivax to chloroquine was first documented in 1989 in Papua, New Guinea and now it is confirmed in Indonesia and Myanmar. Such incidence has only been reported in areas where there is concurrent widespread resistance of P. falciparum to chloroquine the chloroquine resistant strains of P. falciparum was first suspected in Thailand in 1957 and found in patients in Columbia and Thailand in 1960. A high level of chloroquine resistant P. falciparum strain is reported in South East Asia, South Asia, Oceania the Amazon Basin and some coastal areas of South America. In Africa chloroquine resistance was first documented in 1979 in Tanzania and had spread and intensified in the last 20 years. In East Africa and Ethiopia high resistance to chloroquine is experienced but moderate levels of resistance are recorded in Central and Southern Africa. The emergence of multi-drug resistant strains of parasite is also exacerbating the situation. Malaria is now re-emerging in areas where it was previously under control or eradicated e.g., in the Central Asian Republics of Tajikistan and Azerbaijan, and in Korea.

Cost Effective Factor

The direct and indirect costs of malaria in sub-Saharan Africa exceed $2 billion, according to 1997 estimates. According to UNICEF, the average cost for each nation in Africa to implement malaria control programme is estimated to be at least $300,000 a year. This amounts to about six US cents ($0.06) per person for a country of 5 million people.

Characteristics of the New Lead Molecules

The crude extract was prepared by the enzyme-acid hydrolyzing process extracted from Mussel species belonging to family Mytilidae, found in the Ocean waters of Goa, India. The mussels belonging to this family comprise of brown mussel, green mussel and other related mussels. The crude extract prepared by the enzyme-acid hydrolyzing process from a marine organism showed initially a potent anti-malarial activity, at least when examined for in vitro cultures of Plasmodium falciparum in human erythrocytes. This led to an effort towards isolating and characterizing the molecular entity(ies) responsible for anti-malarial activity. An activity-guided fractionation strategy was followed in present research where a variety of chromatographic steps were employed. These, included HPLC using a range of columns (hydrophobic, selective absorption, ion-exchange etc.), preparative thin layer chromatography, selective derivatization and gel filtration chromatography. Selective enrichment of activity was monitored at every step using P. falciparum culture for in vitro studies. This effort has resulted in eventually identifying a compound that independently showed anti-malarial activity. This was followed by structure elucidation of the chemical structure of the compound. The elucidated structure was also independently validated and found potent. The compound has been labeled as PIZ2.

### SUMMARY OF THE INVENTION

In the present study, the compound displays biological activity against the malaria parasite (mouse and human tested). The compound exhibits activity against P. falciparum for in vitro culture assay. Importantly, the compound acts by directly killing parasite, rather than just causing inhibition in their growth. No effect of compound was seen on the host cell in any of our experiments. This confirms that the compounds is non-toxic. The compound also displayed activity against the chloroquine-resistant strain-W2Mef. Important here is the fact that the dose response curves for both 3D7 and W2Mef strains are identical. This strongly suggests that the compound will be equally effective against field isolates of chloroquine-resistant, P. falciparum. Also to be noted here is the fact that PIZ2 acts on W2Mef strain through killing the target.

Finally, both compound shows anti-malarial activity in vivo, at least in the mouse model. Further, blood smears from drug-treated mice also, support that the compound acts through killing the parasite in vivo. Importantly, the bio-activity of the compound is retained when delivered through the oral route, suggesting this as a drug of high promise.

In summary, the compound presents as promising candidate drug for malaria. PI72 is highlighted in this respect because it does not discriminate between chloroquine-sensitive and chloroquine-resistant strains. The results from in vivo experiments, and from those involving oral treatment further confirm these findings.

### OBJECTIVES OF THE INVENTION

The main objective of the present invention is to study the anti-malarial activity of zinc complexes of selected a amino acids wherein the amino acids are selected from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc.

Yet another object of the present invention provides an alternative antimalarial drug of zinc complexes of selected amino acids especially aganist Plasmodium species selected from group of P. vivax, P. ovale, P. malariae, P. falciparum, P. bergei and other known plasmodia.

Still another object of the present invention provides the effect of an alternative antimalarial drug, for drug resistant plasmodium parasites selected from group comprising of P. vivax, P. ovale, P. malariae, P. falciparum, P. bergei and other known plasmodia.

Another object of the present invention provides a method of treatment or prevention of malaria by administering zinc complexes of selected amino acids to mammals, preferably humans, wherein the amino acids are selected from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc

Still another object of the present invention relates to a pharmaceutical composition for prevention or treatment of malaria by administering effective of amount of zinc complexes selected amino acids. Wherein, the amino acids are selected from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc.

### SUMMARY OF THE INVENTION

The present invention relates to the anti-malarial activity of zinc complexes of selected amino acids wherein the amino acids are selected from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc. The compound showed significant inhibition of growth of the human malarial parasite Plasmodium falciparum in RBC cultures in vitro. The compound is lethal to the parasite, but have no effect on the RBCs and mice. The compound has also displayed activity against the chloroquine-resistant strain-W2Mef. The dose response curves for both 3D7 and W2Mef strains are identical which strongly suggested that the compound is equally effective against field isolates of chloroquine-resistant, P. falciparum. The compound acts on W2Mef strain through killing the target.

### BRIEF DESCRIPTION OF THE ACCOMPANYING DRAWINGS/FIGURES

FIG. 1 Growth inhibition of malaria parasite (P. falciparum) with different concentrations of zinc complex of proline.

FIG. 2 The various amino acids screened in the form of their zinc complexes indicate below are as follows:

FIG. 3 The effect of compound on the 3D7 strain of P. falciparum.

FIG. 4 Effect of PIZ2on levels of parasitemia in infected mice

FIG. 5 The effects of oral delivery of PIZ2 on the survival for in vivo studies

FIG. 6 A comparison of the chloroquine-sensitivities of the 3D7 and W2Mef strains of P. falciparum

FIG. 7 Inhibition of CQ-sensitive (3D7) and CQ-resistant (W2Mef) Plasmodium falciparum by PIZ2.

FIG. 8 Effect of PIZ2 on the chloroquine resistent strain-W2Mef

### DETAILED DESCRIPTION OF THE INVENTION

The present invention relates to the use of zinc complexes of select amino acids wherein the amino acids are selected from D or L isomers of proline, lysine, histidine. glycine, arginine and tryptophan or their various hydroxyl, amino, alkyl and carboxyl derivatives and zinc chloride, zinc acetate or other pharmacologically acceptable salts of zinc. The use of the compound comprises administering an effective amount of said compounds for inhibition of growth of the malarial parasite, Plasmodium falciparum. These compounds are lethal to the parasite in RBC cultures but have no effect on the RBCs. The compound has also displayed activity against the chloroquine-resistant strain-W2Mef. The dose response curves for both 3D7 and W2Mef strains are identical which strongly suggested that the compound is equally effective against field isolates of chloroquine-resistant, P. falciparum. The compound acts on W2Mef strain through killing the target.

Accordingly, the main embodiment of the present invention relates to a method of treating and/or preventing malaria said method comprising of administering effective amount of zinc complexes of selected amino acids from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc to mammals, preferably humans, optionally along with acceptable additives, carriers, diluents, solvents, filters, lubricants, excipients, binder or stabilizers.

Another embodiment of the present invention relates to a pharmaceutical composition for prevention or treatment of malaria in mammals, preferably humans said composition comprising of administering effective dose of zinc complexes of selected amino acids from D or L isomers, of proline, lysine, histidine, glycine, arginine and tryptophan or their various hydroxyl, amino, alkyl and carboxyl derivatives and zinc chloride, zinc, acetate or other pharmacologically acceptable salts of zinc to mammals, preferably humans, optionally along with acceptable additives, carriers, diluents, solvents, filters, lubricants, excipients, binder or stabilizers.

Another embodiment of the present invention relates to the zinc complexes of selected amino acids wherein said zinc complexes of selected amino acids are lethal to the malarial plasmodia selected from group comprising of P. vivax, P. ovale, P. malariae, P. falciparum, P. bergei and other known plasmodia.

Another embodiment of the present invention relates to the zinc complexes of selected amino acids wherein said zinc complexes of selected amino acids can be administered along with Phosphono derivatives selected from group comprising of aliphatic mon- and di-carboxylic acids having structural formula: R—COOH, wherein R is PO₃H₂ or CR₁R₂—PO₃H₂, wherein R₁/R₂ are H, OH, COOH or alkyl groups (As filed in U.S. Provisional Patent Application No. 60/512,906, filed on Oct. 20, 2003)

Another embodiment of the present invention relates to the zinc complexes of selected amino acids wherein said zinc complexes of selected amino acids can be administered along with other antimalarial drugs.

Another embodiment of the present invention relates to the other anti-malarial drugs wherein other anti malarial drugs may be selected from group consisting of Chloroquine and its derivatives, Amodiaquine, Sulfadoxine, Pyrimethamine and its derivatives, Proguanil, Mefloquine, Quinine, Halofantrine, Artemisinin, Artemether and Artesunata and their derivatives.

Another embodiment of the present invention relates to the zinc complexes of selected amino acids, wherein zinc complexes of selected amino acids are isolated from extract of Mussel species: belonging to family Mytilidae, found in the Ocean waters of Goa, India.

Another embodiment of the present invention relates to the Mussel species wherein mussel species belonging to family Mytilidae are selected from group consisting of brown mussel green mussel and other related mussels.

Another embodiment of the present invention relates to the zinc complexes of selected amino acids wherein said zinc complexes of selected amino acids are administered in the form of injectables, tablets, capsules, syrup, for the treatment of malaria.

Another embodiment of the present invention relates to thee additives, carriers, diluents, solvents, filters, lubricants, excipients, binder or stabilizers wherein additives, carriers, diluents, solvents, filters, lubricants, excipients, binder or stabilizers maybe selected from group consisting of lactose, mannitol, sorbitol, microcrystalline cellulose, sucrose, sodium citrate, dicalcium phosphate, magnesium stearate, calcium stearate or steorotes, talc, solid polyethylene glycols, sodium lauryl sulphate, cetyl alcohol, glyceryl monostearate or any other acceptable additives, carriers, diluents, solvents, filters, lubricants, excipients, binder or stabilizers of the similar nature alone or in a suitable combination thereof.

Another embodiment of the present invention relates to the zinc complexes of selected amino acids wherein said zinc complexes of selected amino acids are lethal to the parasite, but with no effect on the RBCs.

Another embodiment of the present invention relates to the zinc complexes of selected amino acids wherein said zinc complexes of selected amino acids inhibit the growth of the malarial parasite (Plasmodium falciparum) in RBC cultures.

Another embodiment of the present invention relates to the zinc complexes of selected amino acids wherein zinc complexes of selected amino acids kills the parasites by disintegrating trophozoites.

Another embodiment of the present invention relates to the zinc complexes of selected amino acids wherein about 5 μM to 10 μM of zinc complexes of selected amino acids inhibits growth of malaria parasites.

Another embodiment of the present invention relates to the zinc complexes of selected amino acids wherein about 5 μM to 10 μM of zinc complexes of amino acid proline inhibits growth of P. facliparum by about 100%.

Another embodiment of the present invention relates to the zinc complexes of selected amino acids wherein about 1 mg to 50 mg/kg of zinc complexes of amino acid proline inhibits growth of P. berghei by about 80%.

Another embodiment of the present invention relates to the zinc complexes of selected amino acids wherein about 1 mg to 50 mg/Kg of zinc complexes of amino acid proline inhibits growth of P. yeoeli by about 90%.

Another embodiment of the present invention relates to the zinc complexes of selected amino acids wherein zinc complexes of selected amino acids of about 1 to 50 μM inhibit growth of resistant strain of P. facliparum W2Mef by about 100%, which is not resistant to chloroquin.

The following examples are given by way of illustration of the present invention and therefore should not be construed to limit the scope of the present invention.

### EXAMPLES

### Example 1

### Extraction of Amino Acid Zinc Complexes from Mussel Extract

Mussel hydrolysate was lyophilized to get crude solid from which methanolic extract was obtained by adding 150 ml methanol and stirring for 90 mins at RT. Filtered with filter paper. The filtrate was labeled as AcM. The AcM fraction was subjected to HPLC on a RP-C18 column using a linear gradient of 0-60% B acetonitrile over forty minutes. The peak eluting at void volume (10 mins) was collected and lyophilized. The crude solid was dissolved in 60 ml milliQ water and was fractionated on sephadex-G15 column and eluted with H2O. Fraction 6-11 were pooled and lyophilized and labeled as P2N. P2N was further purified using prep-TLC on silica gel with BAW=4:1.5:1 as the mobile phase. Two fractions labled K-1-1 and K-1-2were obtained after extracting silica gel with 0.01N HCl. Lyophilized to get solid and activity was found in K-1-2. K-1-2 was further sub-fractionated on HSF5 RP column using water as the mobile phase under isocratic condition to get K-1-2/1 and K-1-2/2 and both were giver anti-malaria activity. The fraction K-1-2/1 were found to be Amino Acid-zinc complexes, whereas K-1-2/2 were found to be phosphono derivatives. Both these fractions i.e K-1-2/1 and K-1-2/2 showed anti-malarial activity. Further fraction K-1-2/1 was determined by mass spectrometry confirmed it to be Amino Acid-zinc complex (Zinc-proline Complex). Finally the activity for the isolated compound from the mussle extract was reproduced by commercially available, synthetic L-proline. To confirm that the synthetic and commercially available amino acids would could perform the similar anti-malarial activity as the isolated amino acid-zinc complex following method was followed. The L-Proline-Zn complex mixture was prepared from the commercially available L-Proline and Zinc Chloride: To a solution of 1:15 gms of L-Proline in 10 ml of water (0.01 mol) in a conical flask is added drop-wise a solution of 3.6 gms of Zinc acetate in 10 ml of water (or 2.65 gm of Zinc chloride) (0.02 mol). The mixture is stirred for 10 minutes at room temperature and then gradually heated up to 100 degrees over a period of 20 minutes. The reaction mixture was maintained at 100 degrees for ten minutes and then allowed to cool to room temperature. Aliquotes of this solution were used for the bioassays. Further, the Mass spectrometry between the isolated Amino Acid Zinc complexes from the mussel extract and the commercially available Amino Acid-Zinc complexes when compared matched perfectly to prove the findings from the mussel extract. In other words the mussel extract fraction K-1-2/1 were similar to commercial zinc complexes in their mass spectromretry analysis.

### Example 2

Zinc complex of L-proline was dissolved in normal saline and filter sterilized. The compound was added to the parasite culture at different concentrations ranging from 1- 10 μM. The compound was tested at the indicated doses using the experimental protocol as described below:

Protocol for Testing the Effect of Drug on P. falciparum for In Vitro Studios

The P. falciparum cultures were synchronized at first by sorbitol treatment. The compound of various concentrations was added to the 200 μl of synchronized P. falciparum culture (1% parasitemia). The parasitemia was checked by making Giemsa stained smear after 48 hrs of incubation at 37° C. The growth of P. falciparum was inhibited in dose-dependent manner, where 10 μM concentration yielded >80% inhibition (FIG. 1). The resulting dose-dependent response obtained is shown in Plate 1. The bars represent the percent inhibition, whereas, the blue curve indicates the percentage of parasitemia. From the graph, the concentration required for half-maximal (LD₅₀) inhibition is calculated to be about 7.0 μM.

### Example 3

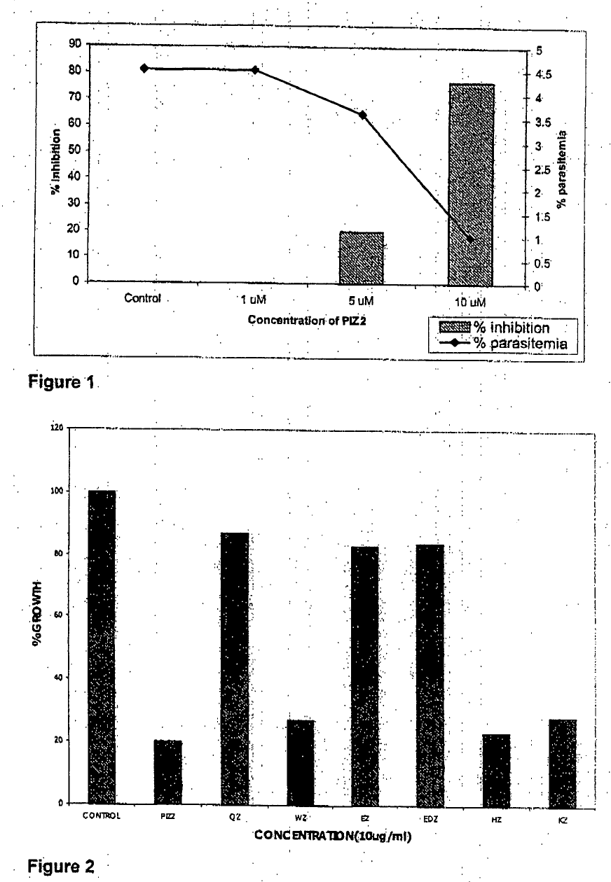

Zinc complexes of L-histidine, L-lysine and L-methionine were dissolved in normal saline and filter sterilized. The compounds were inhibited in dose-dependent manner, where 10 μM concentration yielded ˜85% inhibition (FIG. 2).

### Example 4

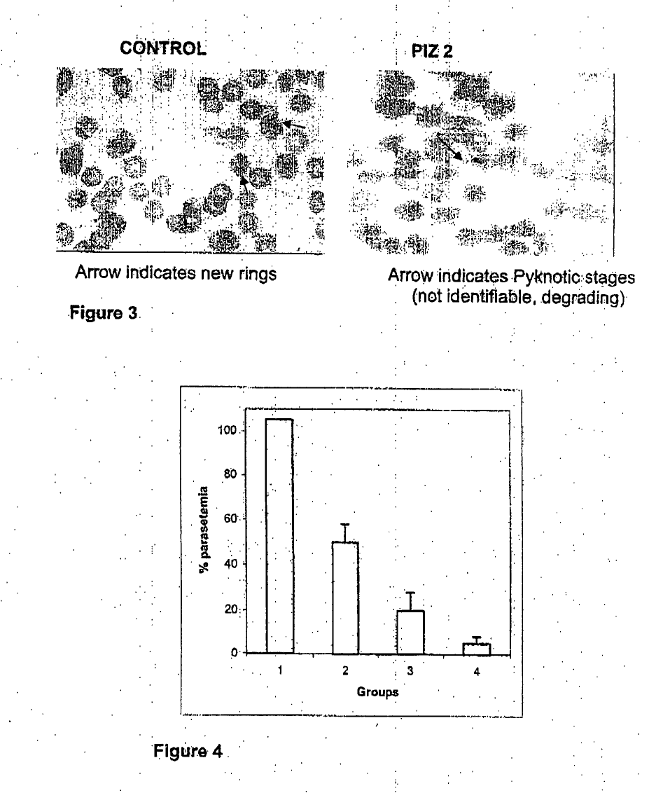

In this experiment, slides were prepared, stained with Giemsa and examined under a light microscope. Representative pictures are show in FIG. 3. A significant disintegration of the parasite is seen upon treatment of cultures with compound PIZ2. This confirms that the action of compound is through direct killing of the parasite.

### Example 5

### Effect of PIZ2 on Levels of Parasitemia in Infected Mice

BALB/c mice (4-6 weeks old) were infected with 10⁵ P. berghei parasites/mouse. PIZ2 was dissolved in saline and filter sterilized. After four days of infection, various concentrations of compound, at the doses of 10 mg/kg (Group 2) and 20 mg/kg (Group 3) body weight was injected to the mice. The control group (Group 1) indicates the group of mice infected with the parasite, but without any drug (i.e. vehicle only). Group 4 represents a group of infected mice treated with Pyrimethamine (at 20 mg/kg body weight) using an identical treatment regimen. Each group contained ten mice and the parasitemia levels—obtained from blood smears—are given as a percentage of that obtained in untreated mice (FIG. 4).

### Example 6

### The Effects of Oral Delivery of NIO-2 on the Parasite Growth for In Vivo Studies

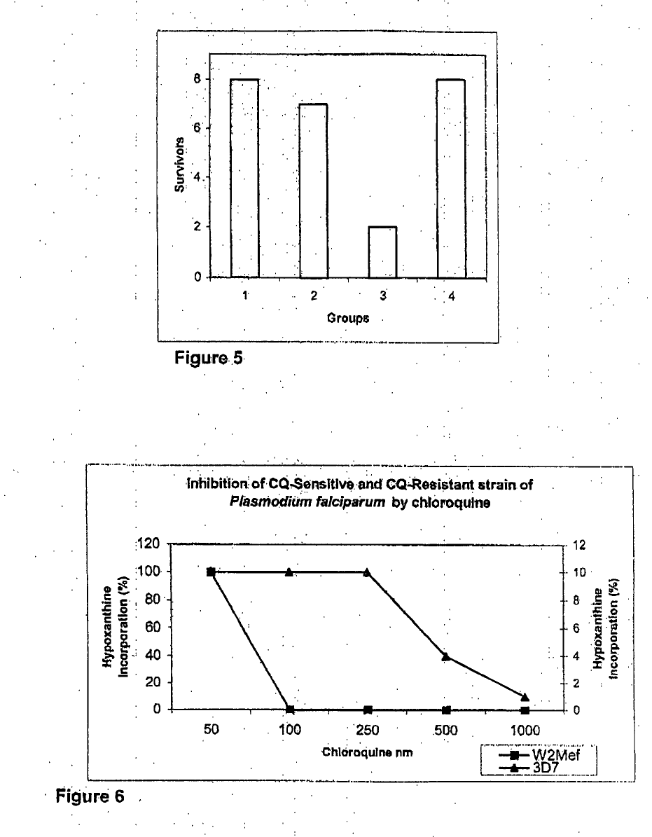

For these experiments, the mouse malaria parasite Plasmodium yeoeli was used as this strain is considered to be closer to human parasite, P. falciparum. Groups of eight mice each were infected with 10⁶ parasites/mouse through an intra-peritoneal injection. Four days later, the parasitemia in these mice was reached between 0.8 and 1%. At this time, the mice in group 2 were orally fed with a dose of 20 mg/kg body weight of PIZ2 (dissolved in water). The drug was given once a day for a total period of six days. The mice were then left alone for an additional period of three days, after which the number of survivors were counted (i.e. at twelve days after infection). The results are shown here in FIG. 5. Group 1 represents a control group of uninfected mice, with no drug given. Group 3 represents the group of infected mice, again with no drug given (i.e. vehicle only). It is evident that the survival of infected mice is substantially increased in group 2 (fed with PIZ2). Group 4 represents a group of infected mice that was treated with combination of 25 microgram Pyrimethamine and 500 microgram of sulfadoxine (per mouse) using the same regimen as for PIZ2. In Group 2, the percentage of survivors was 88% as comapred to Group 3 (% survival: 25%) where no compound was given (FIG. 5)

### Example 7

### Effect of Oral Delivery of PIZ2 on Parasitemia Levels in Infected Mice (Table 1)

Protocol

1) Day 0: Two groups of 8 BALB/c mice infected with 10⁵/Mouse.

2) Day 4: Mice are positive (about 0.5% parasitemia).
One group given PIZ-2 orally at 20 mg/kg body weight (test group). The other group was given vehicle only (Control).

3) Blood smears prepared from individual mice from each group and percent of parasitemia determined.

TABLE 1

| | % Parasitemia | |
|---|---|---|
| | Control | Test |
| Mouse # | group | group |
| 1 | >55% | 4% |
| 2 | >55% | 5% |
| 3 | >55% | 2.5% |
| 4 | >55% | 2% |
| 5 | >55% | 3% |
| 6 | >55% | <0.2% |
| 7 | >55% | 5% |
| 8 | >55% | 6% |

### Example 8

The activity PIZ2 was also compared against a chloroquine resistant strain of P. falciparum. The W2Mef strain was used for these experiments. It was established that this strain indeed was more resistant to chloroquine, at first, and then 3D7 strain was used as the chloroquine-sensitive prototype in our studies. As shown in FIG. 6, the W2Mef strain showed a near sevenfold increase in the IC₅₀ value for chloroquine when compared with the 3D7 strain. The following was the protocol for present experiment.

In the present study, Chloroquine-sensitive (3D7) strain and chloroquine-resistant (W2Mef) strain were used. The cultures were maintained in vitro using culture media consisting of standard RPMI-1640 supplemented with 10% heat inactivated Human Type O⁺ Serum, 3.6% NaHCO₃ at a hematocrit of 4%. All the assays were carried out in 96 well, flat bottom microtitre plates. For the synchronization of cultures, 5% sorbitol was used. The growth of the parasite was measured with incorporation of Hypoxanthine. A 200 μl volume of the culture parasite was added to each well initially. Different antimalarial compounds at varied concentrations were added to the wells. All the compounds were tested on both chloroquine-sensitive and chloroquine resistant strains. The plates were then placed in an airtight chamber and flushed with the gas mixture (5% CO₂, 5% O₂ and 90% N₂). The plates were finally stored in an incubator at 37° C. for 24 hours.

At the end of the incubation period, 5 μl of diluted ³H-Hypoxanthine—to a final concentration of 1 μCi—was added to each well. The plates were then returned to the airtight chamber and then flushed with the above gas mixture and incubated for an additional 24 hours. The plates were then stored at −70° C. until required for detection of incorporated radioactivity. When necessary, the plates were thawed and the cells were harvested onto filter-mats. These filter-mats were then dried and sealed. The filter-mats were subsequently developed using the Betaplate Scint and the incorporated radioactivity determined on the Betaplate Scintillation Counter (FIG. 6).

### Example 9

### The Compound PIZ2 is Equally Active Against a Chloroquine-resistant Strain of P. falciparum

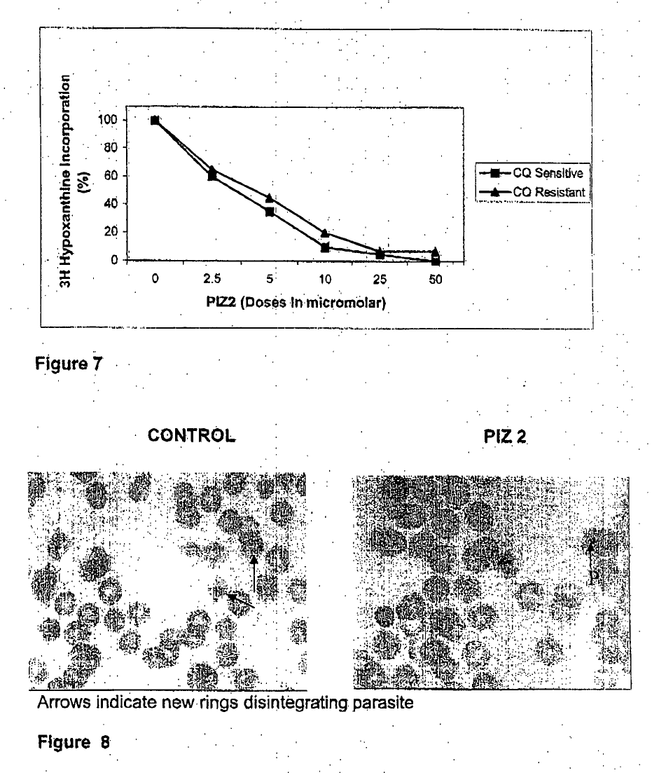

Preliminary experiments indicated that PIZ2 is indeed active against the chloroquine resistant strain-W2Mef. A subsequent dose response analysis confirmed this, and also indicated that the potency of this compound remained identical regardless of whether the target was the chloroqine-sensitive, or the chloroquine-resistant strain of P. falciparum. Representative results from one of three independent experiments are shown in FIG. 7.

### Example 10

The present experiment showed that the PIZ2 also acted on a similar manner on W2Mef through direct killing as it was noticed with 3D7 (FIG. 8).

### ADVANTAGES OF THE PRESENT INVENTION

1) The present active compound for malaria is relatively cheap and can readily be prepared in bulk.

2) It adds to the existing list of anti-malaria compounds, and may be used in conjunction with the other conventional drugs such as chloroquine, mefloquine etc

3) It is also be useful against drug resistant malarial parasite.

### CITED REFERENCES

1) Bradley, D. J. Warhurst, D. C., Br. Med. J., 310, 709-714, 1995.
2) Datta, A. K. and Hood, R. E., Virology, 114, 52-59, 1981.
3) Dorn, A., Stoffel, R., Matile, H., Bubendorf, A., Ridley, R. G., Nature, 374, 269-271, 1995.
4) Malhotra, P, Dasaradhi, P. V. N. Kumar, Amit, Mohammed Asif, Agarwal, N., Bhatnagar, R. K. and Chauhan, V. S., Mol. Microbiol. 45, 1245-1254, 2002
5) McConkey, G. A. Rogers, M. J. and McCutchan, T. F., J. Biol. Chem., 272, 2046-2049, 1997.
6) Murphy, G., Basri, H., Pudromo, Lancet, 341, 96-100, 1993.
7) Oberg, Bo, Pharmac. Ther., 19, 387-415, 1983.
8) Slater, A. F. G., Pharmac. Ther., 57, 203-235, 1993.
9) Tiffert, T., Ginsburg, H., Krugliak, M., Elford, B. C. and Lew, V. L., Proc. Natl. Acad Sci. USA, 97, 331-336, 2000.
10) Wernsdorfer, W. H., Payne, D., Pharmac. Ther., 50, 95-121, 1991.
11) White, N. J., Br. J. Clin. Pharmacol., 34, 1-10, 1992.

1. A method of treating and/or preventing malaria said method comprising of administering effective amount of zinc complexes of selected amino acids from D or L isomers of proline lysine, histidine, glycine, arginine and tryptophan or their various hydroxyl, amino, alkyl and carboxyl derivatives and zinc chloride, zinc acetate or other pharmacologically acceptable salts of zinc to mammals, preferably humans, optionally along with acceptable additives, carriers, diluents, solvents, filters, lubricants, excipients, binder or stabilizers.

2. A method as claimed in claim 1, wherein said zinc complexes of selected amino acids are lethal to the malarial plasmodia selected from group comprising of P. vivax, P. ovale, P. malariae, P. falciparum, P. bergei and other known plasmodia.

3. A method as claimed in claim 1, wherein said zinc complexes of selected amino acids can be administered along with Phosphono derivatives selected from group comprising of aliphatic mon- and di-carboxylic acids having structural formula R—COOH wherein R is PO₃H₂ or CR₁R₂—PO₃H₂, wherein R₁/R₂ are H, OH, COOH or alkyl groups (As filed in, U.S. Provisional Patent Application No. 60/512,906, filed on Oct. 20, 2003)

4. A method as claimed in claim 1, wherein said zinc complexes of selected amino acids can be administered along with other antimalarial drugs.

5. A method as claimed in claim 4 wherein other anti malarial drugs may be selected from group consisting of Chloroquine and its derivatives, Amodiaquine, Sulfadoxine, Pyrimethamine and its derivatives, Proguanil, Mefloquine, Quinine, Halofantrine, Artemisinin, Artemether and Artesunata and their derivatives.

6. A composition as claimed in claim 1, wherein zinc complexes of selected amino acids are isolated from extract of Mussel species belonging to family Mytilidae, found in the Ocean waters of Goa, India.

7. A composition as claimed in claim 6, wherein mussels species belonging to family Mytilidae are selected from group consisting of brown mussel, green mussel and other related mussels.

8. A method as claimed in claim 1 wherein said zinc complexes of selected amino acids are administered in the form of injectables, tablets, capsules, syrup, for the treatment of malaria.

9. A method as claimed in claim 1, wherein additives, carriers, diluents, solvents, filters, lubricants, excipients, binder or stabilizers maybe selected from group consisting of lactose, mannitol sorbitol, microcrystalline cellulose, sucrose, sodium citrate, dicalcium phosphate, magnesium stearate, calcium stearate or steorotes, talc, solid polyethylene glycols, sodium lauryl sulphate, cetyl alcohol, glyceryl monostearate or any other acceptable additives, carriers, diluents, solvents, filters, lubricants, excipients, binder or stabilizers of the similar nature alone or in a suitable combination thereof.

10. A method as claimed in claim 1, wherein said zinc complexes of selected amino acids are lethal to the parasite but with no effect on the RBCs.

11. A method as claimed in claim 1, wherein said zinc complexes of selected amino acids inhibit the growth of the malarial parasite (Plasmodium falciparum) in RBC cultures.

12. A method as claimed in claim 1, wherein zinc complexes of selected amino acids kills the parasites by disintegrating trophozoites.

13. A method as claimed in claim 1, wherein about 5 μM to 10 μM of zinc complexes of selected amino acids inhibits growth of malaria parasites.

14. A method as claimed in claim 1, wherein about 5 μM to 10 μM of zinc complexes of amino acid proline inhibits growth of P. facliparum by about 100%.

15. A method as claimed in claim 1, wherein about 1 mg to 50 mg/kg of zinc complexes of amino acid proline inhibits growth of P. berghei by about 80%.

16. A method as claimed in claim 1, wherein about 1 mg to 50 mg/Kg of zinc complexes of amino acid proline inhibits growth of P. yeoeli by about 90%.

17. A method as claimed in claim 1, wherein zinc complexes of selected amino acids of about 1 to 50 μM inhibit growth of resistant strain of P. facliparum W2Mef by about 100%, which is not resistant to chloroquin.

18. A pharmaceutical composition for prevention or treatment of malaria in mammals, preferably humans said composition comprising of administering effective dose of zinc complexes of selected amino acids from D or L isomers of proline, lysine, histidine, glycine, arginine and tryptophan or their various hydroxy, amino, alkyl and carboxyl derivatives and zinc chloride, zinc acetate or other pharmacologically acceptable salts of zinc to mammals, preferably humans, optionally along with acceptable additives, carriers, diluents, solvents, filters, lubricants, excipients, binder or stabilizers.

19. A composition as claimed in claim 18, wherein said zinc complexes of selected amino acids are lethal to the malarial plasmodia selected from group comprising of P. vivax, P. ovale, P. malariae, P. falciparum, P. bergei and other known plasmodia.

20. A composition as claimed in claim 18, wherein said zinc complexes of selected amino acids can be administered along with Phosphono derivatives selected from group comprising: of aliphatic mon- and di-carboxylic acids having structural formula R—COOH, wherein R is PO₃H₂ or CR₁R₂PO₃H₂, wherein R₁/R₂ are H, OH, COOH or alkyl groups (As filed in U.S. Provisional Patent Application No. 60/512,906, filed on Oct. 20, 2003)

21. A composition as claimed in claim 18, wherein said zinc complexes of selected amino acids can be administered along with other antimalarial drugs.

22. A composition as claimed in claim 21, wherein other anti malarial drugs may be selected from group consisting of Chloroquine and its derivatives, Amodiaquine, Sulfadoxine, Pyrimethamine and its derivatives, Proguanil, Mefloquine, Quinine, Halofantrine, Artemisinin, Artemether and Artesunata and their derivatives.

23. A composition as claimed in claim 18, wherein zinc complexes of selected amino acids are isolated from extract of Mussel species belonging to family Mytilidae, found in the Ocean waters of Goa, India.

24. A composition as claimed in claim 23, wherein mussels species belonging to family Mytilidae are selected from group consisting of brown mussel, green mussel and other related mussels.

25. A composition as claimed in claim 18, wherein said zinc complexes of selected amino acids are administered in the form of injectables, tablets, capsules, syrup, for the treatment of malaria.

26. A composition as claimed in claim 18, wherein additives, carriers, diluents, solvents, filters, lubricants) excipients, binder or stabilizers maybe selected from group consisting of lactose, mannitol, sorbitol, microcrystalline cellulose, sucrose, sodium citrate, dicalcium phosphate, magnesium stearate, calcium stearate or steorotes, talc, solid polyethylene glycols sodium lauryl sulphate, cetyl alcohol, glyceryl monostearate or any other acceptable additives, carriers, diluents, solvents, filters, lubricants, excipients, binder or stabilizers of the similar nature alone or in a suitable combination thereof.

27. A composition as claimed in claim 18, wherein said zinc complexes of selected amino acids are lethal to the parasite, but with no effect on the RBCs.

28. A composition as claimed in claim 18, wherein said zinc complexes of selected a amino acids inhibit the growth of the malarial parasite (Plasmodium falciparum) in RBC cultures.

29. A composition as claimed in claim 18, wherein zinc complexes of selected amino acids kills the parasites by disintegrating trophozoites.

30. A composition as claimed in claim 18, wherein about 5 μM to 10 μM of zinc complexes of selected amino acids inhibits growth of malaria parasites.

31. A composition as claimed in claim 18, wherein about 5 μM to 10 μM of zinc complexes of amino acid proline inhibits growth of P. facliparum by about 100%.

32. A composition as claimed in claim 18, wherein about 1 mg to 50 mg/Kg of zinc complexes of amino acid proline inhibits growth of P. berghei by about 90%.

33. A composition as claimed in claim 18, wherein about 1 mg to 50 mg/Kg of zinc complexes of amino acid proline inhibits growth of P. yeoeli by about 90%.

34. A composition as claimed in claim 21, wherein zinc complexes of selected amino acids of about 1 to 50 μM inhibit growth of resistant strain of P. facliparum W2Mef by about 100%, which is not resistant to chloroquin.
